(19) United States
(12) Patent Application Publication
(10) Pub. No.: US 2005/0255080 A1
(43) Pub. Date: Nov. 17, 2005

(54) Magic bullets and anti - slip

(76) Inventor: Tex - Robert Texas Bailey, Alpine, TX (US)

Correspondence Address: Robert T Bailey, 809 E. Holland Ave., Alpine, TX, 79830, US

(21) Appl. No.: 10/844,464

(22) Filed: May 13, 2004

(51) Int. Cl. A61K 31/74; C11D 1/00

(52) U.S. Cl. 424/78.08; 510/240

(57) ABSTRACT

The long speculated magic bullet that will kill all disease and not hurt humans my be a game that bacteria have been playing for billions of years and what is needed is an Anti-Bullet to protect you from bacteria. Anti-Slip could be an anti-dislodging agent (Anti-Slip) and Anti-Carcinogen and be the Magic Bullet that has been looked for which is really the Anti-Bullet. The refluxed natural rubber could all so be added to mop water to reduce slipping while wet.

The German scientist Paul Ehrlich (1854-1915) came up with the concept in medicine of a magic bullet that would kill bacteria but not harm Humans. He had some success with an arsenic compound that he add natural organic molecules to it, so it wouldn't hurt humans but kill bacteria.

Some bacteria seam to have a magic bullet when they attack other bacteria Under the microscope watching some bacteria they can come up to other bacteria and completely numb other bacteria leaving them void of life without touching them or seeing any visible means to do this (Paramecium). This magic bullet could be that bacteria have could be magnetic, electromagnetic or vibrational.

So basically I feel that bacteria have been playing this magic bullet for billions of years and what we need is a Anti-Bullet, or anti-dislodging agent or maybe Anti-Slip.

Some bacteria adsorb the iron in a Human body and leave the body low on iron. Do they do this to put the magnetic, electromagnetic and vibrational holding properties in their favor and shouldn't to protect yourself from this kind of bacteria take iron supplement or eat meat since it has high iron content?

I have made a new rubber medicine Anti-Slip. I prepare the medicine by heating natural rubber or natural rubber sap in a flask for about an hour and having a small hole in lid that I light with a flame and let burn the exhausting gases (refluxing).

Now smoke is a carcinogen and can cause cancer, and since I am removing this carcinogen from the rubber and let cool, wouldn't the refluxed natural rubber have a tendency to adsorb carcinogens and be Anti-Carcinogenic if taken as medicine in the human body?

This may be why cooking in pressure cooker the food is so good because you are removing carcinogens and the food is Anti-Carcinogenic.

### Preparation of Anti-Slip

I prepare the medicine by heating natural rubber or natural rubber sap in a flask for about an hour and having a small hole in lid that I light with a flame and let burn the exhausting gases (refluxing).

1. The Anti-Slip medicine is an Anti-Carcinogen and can cure cancer.

2. The Anti-Slip refluxed rubber added to mop water can reduce slipping while wet.
